(19) United States
(12) Patent Application Publication
(10) Pub. No.: US 2026/0306722 A1
(43) Pub. Date: Oct. 8, 2026

(54) ANTIMICROBIAL AND/OR RADIOPAQUE BULKING AGENTS

(71) Applicant: Boston Scientific Scimed, Inc., Maple Grove, MN (US)

(72) Inventors: Yen-Hao Hsu, Shrewsbury, MA (US); Nishant Khattar, White Bear Twp, MN (US); Marc Plourde, Worcester, MA (US); Kristen Maynard, Minneapolis, MN (US)

(21) Appl. No.: 19/632,797

(22) Filed: Mar. 30, 2026

Related U.S. Application Data
(60) Provisional application No. 63/782,340, filed on Apr. 2, 2025.

(51) Int. Cl. A61L 31/16; A61L 31/04; A61L 31/14

(52) U.S. Cl. CPC A61L 31/16; A61L 31/048; A61L 31/145; A61L 2300/106; A61L 2300/208; A61L 2300/404

(57) ABSTRACT

According to an aspect, a method includes forming an antimicrobial compound and forming a hydrogel that includes the antimicrobial compound. In implementations, the hydrogel is a cross-linked polyacrylamide-based hydrogel. In some implementations, the antimicrobial compound includes quaternary ammonium. In some implementations, the antimicrobial compound includes a zwitterion-based quaternary ammonium.

### CROSS-REFERENCE TO RELATED APPLICATION

This application claims priority to U.S. Provisional Patent Application No. 63/782,340, filed on Apr. 2, 2025, entitled “ANTIMICROBIAL AND/OR RADIOPAQUE BULKING AGENTS”, the disclosure of which is incorporated by reference herein in its entirety.

### TECHNICAL FIELD

The present invention generally relates to medical devices and materials that are used as bulking agents that include antimicrobial properties and/or radiopaque properties.

### BACKGROUND

Bulking agents are used in the medical industry. Specifically, bulking agents may be placed within the body of a patient to help provide support or structure within the body of a patient. Accordingly, there is a need for bulking agents or other materials that are placed into the body of the patient to include antimicrobial and/or radiopaque properties.

### SUMMARY

According to an aspect, a method includes forming an antimicrobial compound and forming a hydrogel that includes the antimicrobial compound.

In some implementations, the hydrogel is a polyacrylamide-based hydrogel. In some implementations, the hydrogel is a crosslinked polyacrylamide-based hydrogel.

In some implementations, the antimicrobial compound includes quaternary ammonium. In some implementations, the antimicrobial compound includes quaternary ammonium and iodine. In some implementations, the antimicrobial compound has the following formula

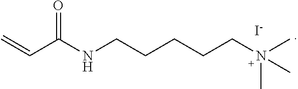

In some implementations, the antimicrobial compound includes a zwitterion-based quaternary ammonium. In some implementation, the antimicrobial compound has the following formula

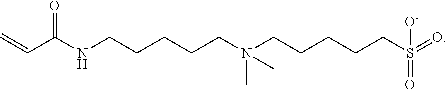

In some implementations, the antimicrobial compound includes quaternary ammonium, the forming the hydrogel includes combining the quaternary ammonium with acrylamide and N,N′-methylenebisacrylamide. In some implementations, the antimicrobial compound includes quaternary ammonium and iodine, the forming the hydrogel includes combining the quaternary ammonium with acrylamide and N,N′-methylenebisacrylamide.

In some implementations, the antimicrobial compound has the following formula

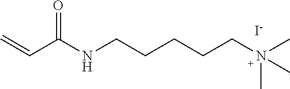

and the forming the hydrogel includes combining the compound of the following formula

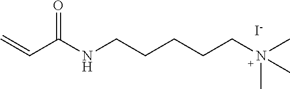

with a compound having the following formula

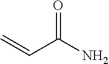

and a compound having the following formula

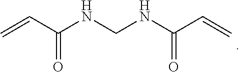

In some implementations, the antimicrobial compound includes a zwitter-based quaternary ammonium, the forming the hydrogel includes combining the zwitter-based quaternary ammonium with acrylamide. In some implementations, the antimicrobial compound includes a zwitter-based quaternary ammonium, the forming the hydrogel includes combining the zwitter-based quaternary ammonium with acrylamide and N,N′-methylenebisacrylamide.

In some implementations, the antimicrobial compound has the following formula

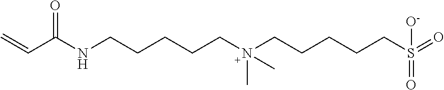

and the forming the hydrogel includes combining the compound of the following formula

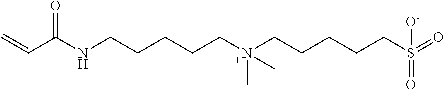

with a compound having the following formula

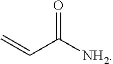

In some implementations, the antimicrobial compound has the following formula

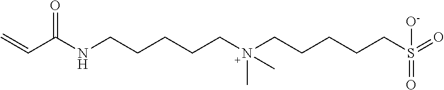

and the forming the hydrogel includes combining the compound of the following formula

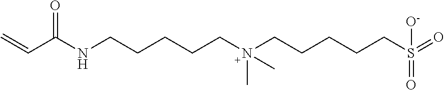

with a compound having the following formula

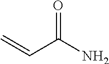

and a compound having the following formula

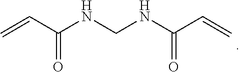

According to another aspect, a composition includes a crosslinked polyacrylamide-based hydrogel, the hydrogel including an antimicrobial compound. In some implementations, the antimicrobial compound includes quaternary ammonium. In some implementations, the antimicrobial compound includes quaternary ammonium and iodine.

In some implementations, the antimicrobial compound has the following formula

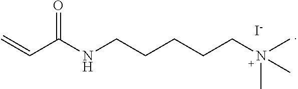

In some implementations, the antimicrobial compound includes a zwitterion-based quaternary ammonium.

### BRIEF DESCRIPTION OF THE DRAWINGS

FIG. 1 illustrates a bulking agent being inserted into a body of a patient according to an implementation.

FIG. 2 illustrates a bulking agent disposed within a body of a patient according to an implementation.

FIG. 3 schematically illustrates a method or pathway for forming a quaternary ammonium functionalized acrylamide.

FIG. 4 schematically illustrates a method or pathway for forming a crosslinked polyacrylamide-based hydrogel.

FIG. 5 schematically illustrates a method or pathway for forming a zwitterion-based quaternary ammonium functionalized acrylamide.

### DETAILED DESCRIPTION

Detailed implementations are disclosed herein. However, it is understood that the disclosed implementations are merely examples, which may be embodied in various forms. Therefore, specific structural and functional details disclosed herein are not to be interpreted as limiting, but merely as a basis for the claims and as a representative basis for teaching one skilled in the art to variously employ the embodiments in virtually any appropriately detailed structure. Further, the terms and phrases used herein are not intended to be limiting, but to provide an understandable description of the present disclosure.

The terms “a” or “an,” as used herein, are defined as one or more than one. The term “another,” as used herein, is defined as at least a second or more. The terms “including” and/or “having”, as used herein, are defined as comprising (i.e., open transition). The term “coupled” or “moveably coupled,” as used herein, is defined as connected, although not necessarily directly and mechanically.

The present invention relates to medical devices such as bulking materials or bulking agents that may be placed within a body of a patient. In some implementations, the bulking agent may be placed near that bladder of the patient (such as near the junction of the bladder and the urethra). In such cases, the bulking agent may help support or provide structure to the body near the bladder to help treat urinary incontinence. In other implementations, the medical device or bulking agent may be placed at a different location within the body of the patient.

In some implementations, the medical device or bulking agent includes antimicrobial portions or properties. In some implementations, the medical device or bulking agent includes radiopaque portions or properties. In some implementations, the medical device or bulking agent includes both antimicrobial portions or properties and radiopaque portions or properties.

Various embodiments, aspects, or implementations of the medial devices, bulking agents, or compounds are described herein. They should be considered as exemplary and should not be used to limit the scope of the invention.

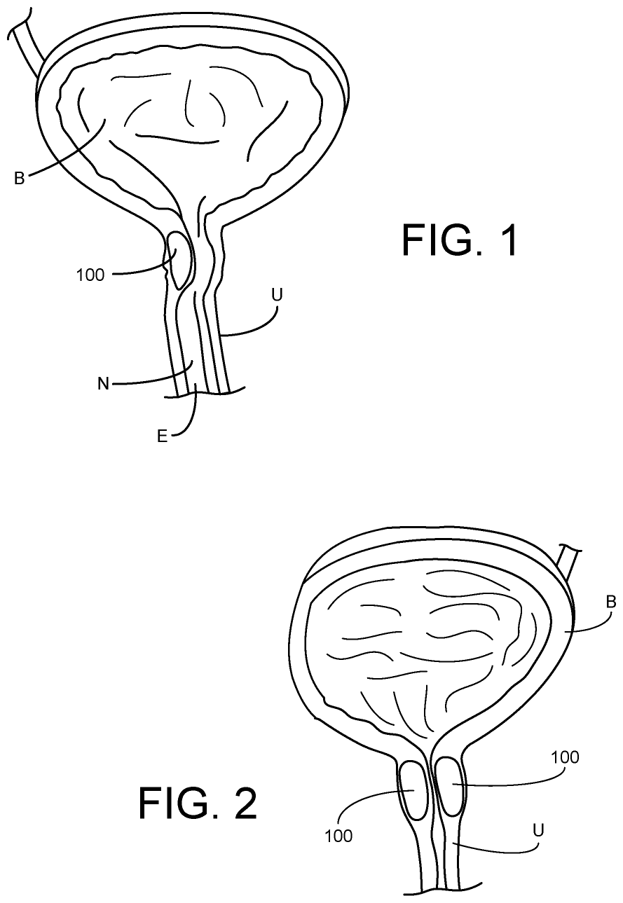

FIG. 1 illustrates a medical device, such as a bulking agent or composition 110 being inserted into a body of a patient according to an implementation. FIG. 2 illustrates the medical device, bulking agent, or composition 110 disposed within the body of the patient.

In the illustrated implementation, the medical device, bulking agent, or composition 110 is inserted or delivered into the body of the patient using a needle N. For example, in some implementations, the medical device, the bulking agent, or composition 110 may be inserted into the body of the patient using a needle N and an endoscope or a short ureteroscope. In other implementations, the medical device, the bulking agent, or composition 110 may be inserted or delivered into the body of the patient using a different delivery device or delivery method.

In the illustrated implementation, the medical device, the bulking agent, or composition 110 is disposed within the body of the patient near or proximate a bladder B of the patient. More specifically, the medical device, the bulking agent, or the composition 110 is disposed or placed near the junction of the bladder B and the urethra U of the patient. As best illustrated in FIG. 2, the medical device, the bulking agent, or the composition 110 may be placed is a couple of locations near the junction of the bladder B and the urethra U of the patient or may be placed such that it surrounds or substantially surrounds the junction of the bladder B and the urethra U of the patient.

In some implementations, the medical device, bulking agent, or composition 110 is configured to provide structure or support to the body of the patient. In some implementations, the medical device, bulking agent, or composition 110 is configured to provide support to the body of the patient such that urinary continence of the patient is improved. In other implementations, the medical device, bulking agent, or composition 110 is disposed in a different portion of the body and has a different therapeutic function within the body of the patient.

In some implementations, the medical device, bulking agent, or composition 110 includes a component or portion that has antimicrobial properties. For example, in some implementations, the medical device, bulking agent, or composition 110 has antimicrobial properties and as such is configured to repel bacteria, kill bacteria, or both. Accordingly in some implementations, the antimicrobial properties help prevent device-associated infections. In some implementations, the medical device, bulking agent, or composition 110 includes an antimicrobial compound. In some implementations, the medical device, bulking agent, or composition 110 is a hydrogel that includes an antimicrobial compound.

In some implementations, the medical device, bulking agent, or composition 110 includes a component or portion that has radiopaque properties. For example, in some implementations, the radiopaque portion is configured to be visible using an x-ray machine or other device. In such implementations, a physician may view the medical device, bulking agent, or composition 110 while it is within the body of the patient either during implantation of the device 110 or after implantation of the device 110 or both to confirm placement or location of the medical device, bulking agent, or composition 110. In some implementations, the medical device, bulking agent, or composition 110 is a hydrogel that includes a radiopaque compound.

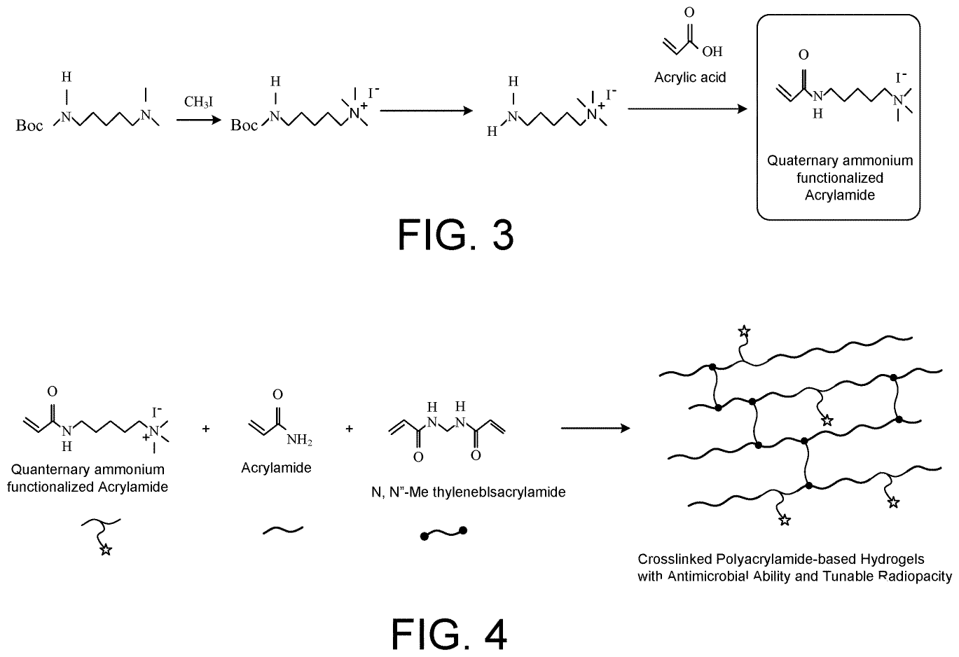

FIG. 3 schematically illustrates a method or pathway for forming an antimicrobial and radiopaque compound. Specifically, FIG. 3 schematically illustrates a method or pathway for forming a quaternary ammonium functionalized acrylamide.

In one implementation, at step one, commercially available boc-protected tertiary amino compound, tert-butyl N-[5-(dimethylamino) pentyl]carbamate (CAS #1698868-24-4) is dissolved in polar aprotic solvent (such as DMSO (dimethyl sulfoxide), DMF (dimethylformamide), or acetonitrile) and iodo methane is added sequentially. After homogeneously (or substantially homogeneously) mixed, the mixture is stirred over night at 50 degrees C. After that, the reaction system is cooled and crude mixture is poured into cold ether, the targeted product is precipitated out and further rinsed with cold ether 3 times to obtain the compound 1, boc-protected quaternary ammonium salt.

In some implementations, this reaction can be performed at neat condition which is “solvent-free.”

In some implementations, the starting material can be any compound with one amine end group that is protected by Boc (or other protection group) and another end group that includes a tertiary amine.

In some implementations, a different compound may be used instead of the iodo methane. For example, Ant iodinated aromatic rings system with alkyl chain and halogen end groups (I, Br, Cl) for further substitution reaction through Sₙ2. Specifically in some implementations, one of the below compounds may be used instead of the iodo methane.

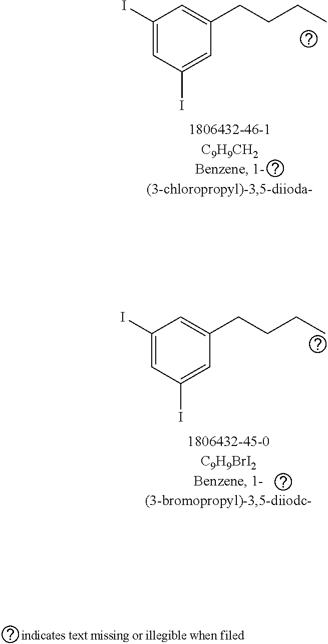

In some implementations, at step two, the 1, boc-protected quaternary ammonium salt compound is further reacted with TFA (trifluoroacetic acid) in DCM (dichloromethane) and stirred for about 3 hours to undergo Boc-deprotection reaction. After reaction is completed, the crude material is further precipitated out in hexane or ether and further titrated to be neutral to obtain the intermediate compound.

In some implementations, the mixture may be stirred for more than 3 hours. In other implementations, the mixture maybe stirred for less than 3 hours.

In some implementations, the Boc-deprotection reaction can be performed in HCl in dioxane (4M) or in any acidic conditions.

As illustrated, at step 3, commercially available acrylic acid (CAS #79-10-7) is mixed with the product of step 2 in any aprotic solvents (such as but not limited to DCM (dichloromethane) or DMF (dimethylformamide) or DMSO (dimethyl sulfoxide) or ACN (acetonitrile) with a coupling agent (such as but not limited to DIC (diisopropylcarbodiimide) or DCC (dicyclohexylcarbodiimide)) and stirred for 12 hours to undergo an amide coupling reaction. In other implementations, the mixture may be stirred for more than 12 hours or for less than 12 hours. After stirring the mixture, the crude product is precipitated out in cold ether to obtain the desired final product, quaternary ammonium iodinated acrylamide.

The final product, quaternary ammonium iodinated acrylamide is a compound that has antimicrobial properties and radiopaque properties. As illustrated in FIG. 4, the quaternary ammonium iodinated acrylamide may be used to form or may be otherwise incorporated into a hydrogel.

As illustrated in FIG. 4, acrylamide monomers (acrylamide and the quaternary ammonium iodinated acrylamide in the illustrated implementation) are dissolved in water, then a crosslinking agent such as N,N′-methylene bisacrylamide (bis-acrylamide) is added. Then a free radical initiator (such as ammonium persulfate (APS)) and a catalyst (such as TEMED (tetramethylethylenediamine)) are added to initiate the polymerization.

In some implementations, the reaction conditions, such as temperature, can be varied depending on the chosen free radical initiator. For example, in the illustrated implementation, the temperature could be between room temperature and 60 degrees C. In other implementations, the temperature is greater than 60 degrees C.

In some implementations, the molar ratio of the acrylamide and the quaternary ammonium iodinated acrylamide can be varied depending on the desirable radiopacity. For example, in some implementations, a higher molar ratio of the quaternary ammonium iodinated acrylamide will result in higher radiopacity of the final hydrogel product.

In some implementations, the crosslinking density can be varied depending on the amount of the crosslinker, N,N′-methylene bis-acrylamide (bis-acrylamide) that is used. For example, in some implementations, the more of the crosslinker that is used the more crosslinking there will be in the final hydrogel product.

In some implementations, solid content can be varied depending on water uptake. The water uptake is crosslinking density dependent. Accordingly, in some implementations, higher crosslinking density of the final product will have lower water uptake.

In some implementations, the final product, the crosslinked hydrogel, can be ground into a desired particle size. In some implementations, the particles of the final product may be used as an injectable gel (or a gel configured to be injected into a body of a patient).

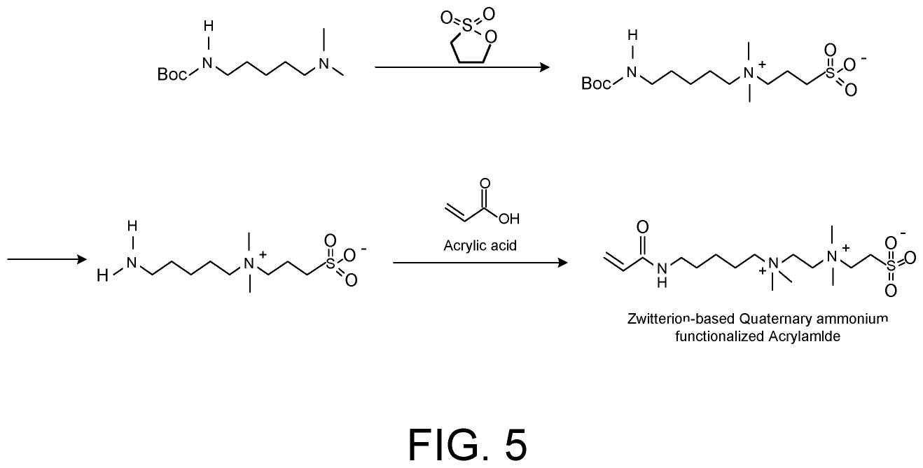

FIG. 5 schematically illustrates a method or pathway for forming an antimicrobial and radiopaque compound. Specifically, FIG. 5 schematically illustrates a method or pathway for forming a zwitterion-based quaternary ammonium functionalized acrylamide.

As best illustrated in FIG. 5, commercially available boc-protected tertiary amino compound, tert-butyl N-[5-(dimethylamino) pentyl]carbamate (CAS #1698868-24-4) is dissolved in polar aprotic solvent (such as DMSO (dimethyl sulfoxide), DMF (dimethylformamide), ACN (acetonitrile) and sultone (such as 1,3-propane sultone) is added. After homogeneously (or substantially homogeneously) mixed, the mixture is stirred over night at 50 C. The reaction system is then cooled and the crude mixture is poured into cold ether. The targeted product is precipitated out and further rinsed with cold ether 3 times to obtain pure or substantially pure product compound 5, boc-protected quaternary ammonium salt with zwitterion form.

In some implementations, this reaction can be performed at neat condition which is “solvent-free.” In some implementations, any functionalized sultone can be used.

At step 2, the intermediate compound is further reacted with TFA (trifluoroacetic acid) in DCM (dichloromethane) and stirred for 3 hours to undergo a Boc-deprotection reaction. After reaction is complete, the crude product is further precipitated out in hexane or ether and further titrated to be neutral to obtain the second intermediate compound.

In some implementations, the Boc-deprotection reaction conditions can be HCl in dioxane (4M) or any other acid conditions.

In the illustrated implementation, commercially available acrylic acid (CAS #79-10-7) is mixed with the second intermediate compound in any aprotic solvents (such as DCM (dichloromethane) or DMF (dimethylformamide), or DMSO (dimethyl sulfoxide) or ACN (acetonitrile) with a coupling agent (such as DIC (diisopropylcarbodiimide) or DCC (dicyclohexylcarbodiimide)) and stirred for 12 hours to undergo an amide coupling reaction. The crude product is precipitated out in cold ether to obtain the desired compound, the final product, quaternary ammonium zwitterionic acrylamide.

The final product, quaternary ammonium zwitterionic acrylamide is a compound that may have antimicrobial properties. As described above with respect to the quaternary ammonium iodinated acrylamide, the quaternary ammonium zwitterionic acrylamide may be used to form or may be otherwise incorporated into a hydrogel.

For example, acrylamide monomers (acrylamide and the quaternary ammonium zwitterionic acrylamide) are dissolved in water, then a crosslinking agent such as N,N′-methylene bisacrylamide (bis-acrylamide) is added. Then a free radical initiator (such as ammonium persulfate (APS)) and a catalyst (such as TEMED (tetramethylethylenediamine)) are added to initiate the polymerization.

In some implementations, the reaction conditions, such as temperature, can be varied depending on the chosen free radical initiator. For example, the temperature could be between room temperature and 60 degrees C. In other implementations, the temperature is greater than 60 degrees C.

In some implementations, the molar ratio of the acrylamide and the quaternary ammonium zwitterionic acrylamide can be varied depending on the desirable radiopacity. For example, in some implementations, a higher molar ratio of the quaternary ammonium iodinated acrylamide will result in higher radiopacity of the final product.

In some implementations, the crosslinking density can be varied depending on the amount of the crosslinker, N,N′-methylene bis-acrylamide (bis-acrylamide) that is used. For example, in some implementations, the more of the crosslinker that is used the more crosslinking there will be in the final hydrogel product.

In some implementations, solid content can be varied depending on water uptake. The water uptake is crosslinking density dependent. Accordingly, in some implementations, higher crosslinking density of the final hydrogel product will have lower water uptake.

In some implementations, the final product, the crosslinked hydrogel, can be ground into a desired particle size. In some implementations, the particles of the final product may be used as an injectable gel (or a gel configured to be injected into a body of a patient).

While certain features of the described implementations have been illustrated as described herein, many modifications, substitutions, changes and equivalents will now occur to those skilled in the art. It is, therefore, to be understood that the appended claims are intended to cover all such modifications and changes as fall within the scope of the embodiments.

What is claimed is:

1. A method, comprising:
forming an antimicrobial compound; and
forming a hydrogel that includes the antimicrobial compound.

2. The method of claim 1, wherein the hydrogel is a polyacrylamide-based hydrogel.

3. The method of claim 1, wherein the hydrogel is a crosslinked polyacrylamide-based hydrogel.

4. The method of claim 1, wherein the antimicrobial compound includes quaternary ammonium.

5. The method of claim 1, wherein the antimicrobial compound includes quaternary ammonium and iodine.

6. The method of claim 1, wherein the antimicrobial compound has the following formula

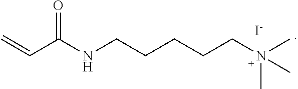

7. The method of claim 1, wherein the antimicrobial compound includes a zwitterion-based quaternary ammonium.

8. The method of claim 1, wherein the antimicrobial compound has the following formula

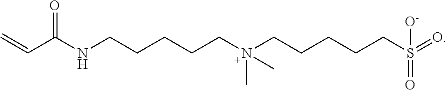

9. The method of claim 1, wherein the antimicrobial compound includes quaternary ammonium, the forming the hydrogel includes combining the quaternary ammonium with acrylamide and N,N′-methylenebisacrylamide.

10. The method of claim 1, wherein the antimicrobial compound includes quaternary ammonium and iodine, the forming the hydrogel includes combining the quaternary ammonium with acrylamide and N,N′-methylenebisacrylamide.

11. The method of claim 1, wherein the antimicrobial compound has the following formula 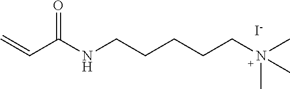
and the forming the hydrogel includes combining the compound of the following formula 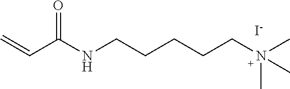
with a compound having the following formula 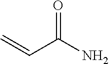
and a compound having the following formula

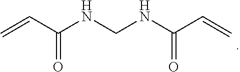

12. The method of claim 1, wherein the antimicrobial compound includes a zwitter-based quaternary ammonium, the forming the hydrogel includes combining the zwitter-based quaternary ammonium with acrylamide.

13. The method of claim 1, wherein the antimicrobial compound includes a zwitter-based quaternary ammonium, the forming the hydrogel includes combining the zwitter-based quaternary ammonium with acrylamide and N,N′-methylenebisacrylamide.

14. The method of claim 1, wherein the antimicrobial compound has the following formula 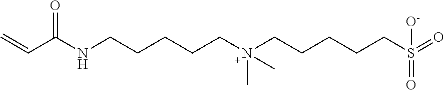
and the forming the hydrogel includes combining the compound of the following formula 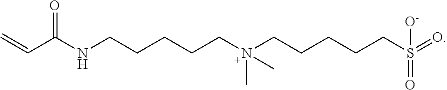
with a compound having the following formula

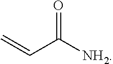

15. The method of claim 1, wherein the antimicrobial compound has the following formula 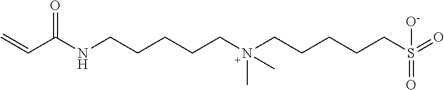
and the forming the hydrogel includes combining the compound of the following formula 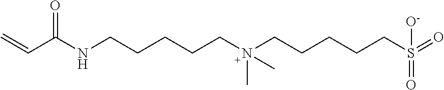
with a compound having the following formula 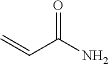
and a compound having the following formula

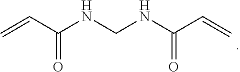

16. A composition, comprising:
a crosslinked polyacrylamide-based hydrogel, the hydrogel including an antimicrobial compound.

17. The composition of claim 16, wherein the antimicrobial compound includes quaternary ammonium.

18. The composition of claim 16, wherein the antimicrobial compound includes quaternary ammonium and iodine.

19. The composition of claim 16, wherein the antimicrobial compound has the following formula

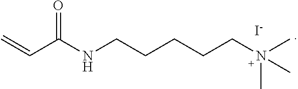

20. The composition of claim 16, wherein the antimicrobial compound includes a zwitterion-based quaternary ammonium.
